(19) United States
(12) Patent Application Publication
(10) Pub. No.: US 2013/0231371 A1
(43) Pub. Date: Sep. 5, 2013

(54) SPOT-ON PESTICIDE COMPOSITION CONTAINING A PYRETHROID AND MACROCYCLIC LACTONE

(71) Applicant: SERGEANT'S PET CARE PRODUCTS, INC., Omaha, NE (US)

(72) Inventors: Larry Nouvel, Plano, TX (US); Donald W Kelley, Athens, TX (US)

(73) Assignee: Sergeant's Pet Care Products, Inc., Omaha, NE (US)

(21) Appl. No.: 13/857,795

(22) Filed: Apr. 5, 2013

Related U.S. Application Data
(63) Continuation-in-part of application No. 13/206,885, filed on Aug. 10, 2011, now Pat. No. 8,455,545.
(60) Provisional application No. 61/620,693, filed on Apr. 5, 2012.

(51) Int. Cl. A61K 31/365; A61K 31/415; A61K 31/08; A61K 31/455; A61K 31/277; A61K 31/231

(52) U.S. Cl. CPC A61K 31/365; A61K 31/277; A61K 31/231; A61K 31/08; A61K 31/455; A61K 31/415
USPC 514/337; 514/450; 514/404

(57) ABSTRACT

A spot-on pesticide composition for animals, specifically mammals, namely dogs, which composition comprises a combination of a pyrethroid and a macrocyclic lactone selected from avermectin, ivermectin, selamectin, moxidectin, milbemycin and any combination thereof, and optionally fipronil and/or an insect growth regulator, in doses and proportions which are parasiticidally effective against a variety of insects and pests, and in a formulation which is convenient for local application to the animal's skin, preferably localized over a small surface area.

### CROSS-REFERENCE TO RELATED APPLICATIONS

This application claims priority to U.S. Provisional Application Ser. No. 61/620,693 filed on Apr. 5, 2012 and is a continuation-in-part of U.S. Nonprovisional application Ser. No. 13/206,885 filed on Aug. 10, 2011, the disclosures of which are hereby incorporated by reference in their entirety.

### FIELD OF THE INVENTION

The present invention relates to a spot-on or pour-on pesticide composition comprising a pyrethroid and a macrocyclic lactone selected from an avermectin, ivermectin, selamectin, moxidectin, milbemycin, and any combination thereof, which combinations are useful in the treatment or prevention of heartworm infections, tick and flea infestations, flea allergy dermatitis, ear mites and sarcoptic mange in animals, specifically dogs. Various embodiments of the present invention may further additionally include fipronil and/or an insect growth regulator.

### BACKGROUND OF THE INVENTION

Traditional products for the treatment or prevention of heartworm infections, tick and flea infestations, flea allergy dermatitis, ear mites and sarcoptic mange of animals include shampoo treatments, insecticidal collars, orally ingested treatments, compositions designed to treat an animal's environment, spot-on treatments, and the like. Different treatment forms offer unique benefits and drawbacks; however, the majority offer substantial disadvantages. For instance, shampoo treatments require that the treatment be applied over the entire surface of the animal and subsequently rinsed off, which is typically unpleasant for both the animal and the owner and only provides a short-term, transient treatment. Insecticidal collars require the animal to physically wear the collar for a period of time often lasting several months, which is uncomfortable and burdensome to the animal. Additional, treatments administered orally tend to increase the possibility of side effect and are more difficult to administer to the animal. Alternatively, treatment of the animal's surroundings and habitat is often undesirable due to the fact that the treatment may cause discoloration of furniture, carpet, bedding, etc., and may also produce unpleasant odors. Thus, it is desirable to have a spot-on treatment that can be applied to the animal in smaller portions, while maintaining treatment efficacy across the entire body surface of the animal.

Spot-on compositions that have been previously developed incorporate a multitude of pesticide agents. Common agents include arylpyrazole derivatives, insect growth regulators, pyrethroids, nodulisporic acid derivatives, neonicotinoids, formamides, avermectins, and the like. All of the compounds listed herein have different mechanisms of action, and accordingly treat and prevent infestation in different manners. Consequently, the various compounds also have a variety of different adverse effects associated with treatment. The various agents may be combined in a variety of concentrations. Generally, higher concentrations of the active components result in higher pest kill rates, and more successful treatments; however, the use of higher concentrations of the active components are more expensive to make and result in a greater likelihood that the animal will suffer adverse effects from treatment. Adverse effects of treatments include skin discoloration, local hair loss, itching, redness, excessive salivation, and in certain cases, neurotoxicity. In addition, safety issues for using these compounds in high concentration in puppies as young as 6 weeks, breeding and nursing animals are also of concern.

The spot-on treatments known within the art generally have a prolonged period of action before the active ingredient(s) effectively eliminates the target pest. For instance, insect growth regulators (i.e. juvenile hormone mimetics) exterminate target pests by inhibiting the development of immature pests so that they are not able to reproduce. Even though the insect growth regulators are effective in ultimately controlling the pest infestation, additional time is required to kill all pests, which leads to additional time in which the animal host, as well as all other animals and humans, must suffer the effects of the infestation. Even quick-acting agents, such as the arylpyrazole derivative known as fipronil, which causes hyperexcitation of the pest leading to its death, have a prolonged onset of action. Generally, it may take multiple hours for quick-acting agents to provide symptomatic relief to the host animal.

Therefore, given the limitations of the prior art, it would be desirable to have a spot-on pesticide treatment that utilizes low concentrations of known chemicals so as to minimize the risk of adverse effects, has a high pest kill rate, and has an improved kill rate, preferably within the first hour of treatment.

### SUMMARY OF THE INVENTION

The invention relates to novel spot-on compositions for treating and preventing of heartworm infections, tick and flea infestations, flea allergy dermatitis, ear mites and sarcoptic mange in animal, as well as a method of killing pests comprising applying the compositions to a host animal, specifically a mammal. The spot-on insecticidal compositions of the current invention comprise pyrethroid and macrocyclic lactone components, and may additionally include fipronil and/or an insect growth regulator. The macrocyclic lactone may be selected from the group consisting of avermectin, ivermectin, selamectin, moxidectin, milbemycin, and any combination thereof. It has further been discovered that these novel combinations of active components not only have a higher and faster kill rate of ectoparasites (e.g. fleas, ticks, mites, etc.) than treatment with a pyrethroid and/or a macrocyclic lactone alone with or without an additional pesticide, but the combination of active components also treats and prevents endoparasite infections (e.g., heartworm). The compositions of the present invention further comprise a paresthesia-reducing agent selected from the group consisting of purified diethylene glycol monoethyl ether, tocopherol nicotinate, and combinations thereof, wherein the paresthesia-reducing agent is present in an amount effective to reduce or eliminate the paresthesia caused by the pyrethroid to the animal.

One embodiment of the current invention relates to a spot-on composition comprising between about 0.25% and about 60% (w/w) pyrethroid, between about 0.01% and about 10% (w/w) macrocyclic lactone, and an effective amount of a paresthesia-reducing agent selected from the group consisting of purified diethylene glycol monoethyl ether, tocopherol nicotinate, and combinations thereof to reduce the paresthesia caused by the pyrethroid to an animal. More specifically, this embodiment of the spot-on composition includes between about 10% and about 45% (w/w) pyrethroid, between about 0.1% and about 8% (w/w) macrocyclic lactone, and an effective amount of a paresthesia-reducing agent selected from the group consisting of purified diethylene glycol monoethyl ether, tocopherol succinate, tocopherol nicotinate, and combinations thereof. Most specifically, the first embodiment of the present invention includes between about 15% and about 30% (w/w) pyrethroid, between about 0.2% and about 6% (w/w) macrocyclic lactone, and an effective amount of a paresthesia-reducing agent selected from the group consisting of purified diethylene glycol monoethyl ether, tocopherol succinate, tocopherol nicotinate, and combinations thereof. The macrocyclic lactone preferred in this embodiment is ivermectin.

A second embodiment of the current invention relates to a spot-on composition comprising between about 0.25% and about 60% (w/w) pyrethroid, between about 0.01% and about 10% (w/w) macrocyclic lactone, between about 1% and about 20% (w/w) insect growth regulator, and a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce the paresthesia caused by the pyrethroid to an animal. More specifically, this embodiment of present composition includes between about 10% and about 45% (w/w) pyrethroid, between about 0.1% and about 8% (w/w) macrocyclic lactone, between about 4% and about 15% (w/w) insect growth regulator, and an effective amount of a paresthesia-reducing agent selected from the group consisting of purified diethylene glycol monoethyl ether, tocopherol succinate, tocopherol nicotinate, and combinations thereof. Most specifically, the spot-on composition of the present invention includes between about 15% and about 30% (w/w) pyrethroid, between about 0.2% and about 6% (w/w) macrocyclic lactone, between about 7% and about 11% (w/w) insect growth regulator, and an effective amount of a paresthesia-reducing agent selected from the group consisting of purified diethylene glycol monoethyl ether, tocopherol succinate, tocopherol nicotinate, and combinations thereof. The macrocyclic lactone preferred in this embodiment is ivermectin.

A third embodiment of the current invention relates to a spot-on composition comprising between about 0.25% and about 60% (w/w) pyrethroid, between about 0.01% and about 10% (w/w) macrocyclic lactone, between about 1% and about 20% (w/w) insect growth regulator, and a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce the paresthesia caused by the pyrethroid to an animal. More specifically, this embodiment of present composition includes between about 10% and about 45% (w/w) pyrethroid, between about 1.5% and about 6% (w/w) macrocyclic lactone, between about 4% and about 15% (w/w) insect growth regulator, and an effective amount of a paresthesia-reducing agent selected from the group consisting of purified diethylene glycol monoethyl ether, tocopherol succinate, tocopherol nicotinate, and combinations thereof. Most specifically, the spot-on composition of the present invention includes between about 15% and about 30% (w/w) pyrethroid, between about 2% and about 4% (w/w) macrocyclic lactone, between about 7% and about 11% (w/w) insect growth regulator, and an effective amount of a paresthesia-reducing agent selected from the group consisting of purified diethylene glycol monoethyl ether, tocopherol succinate, tocopherol nicotinate, and combinations thereof. The macrocyclic lactone preferred in this embodiment is moxidectin.

A fourth embodiment of the current invention relates to a spot-on composition comprising between about 0.25% and about 60% (w/w) pyrethroid, between about 0.01% and about 10% (w/w) of a combination of two or more macrocyclic lactones, between about 1% and about 20% (w/w) insect growth regulator, and a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce the paresthesia caused by the pyrethroid to an animal. More specifically, this embodiment of present composition includes between about 10% and about 45% (w/w) pyrethroid, between about 0.05% and about 10% (w/w) of a combination of two or more macrocyclic lactones, between about 4% and about 15% (w/w) insect growth regulator, and an effective amount of a paresthesia-reducing agent selected from the group consisting of purified diethylene glycol monoethyl ether, tocopherol succinate, tocopherol nicotinate, and combinations thereof. Most specifically, the spot-on composition of the present invention includes between about 15% and about 30% (w/w) pyrethroid, between about 0.05% and about 10% (w/w) of a combination of two or more macrocyclic lactones, between about 7% and about 11% (w/w) insect growth regulator, and an effective amount of a paresthesia-reducing agent selected from the group consisting of purified diethylene glycol monoethyl ether, tocopherol succinate, tocopherol nicotinate, and combinations thereof. The macrocyclic lactones preferred in this embodiment are selected from the group consisting of ivermectin, selamectin, moxidectin, milbemycin, and combinations thereof.

A fifth embodiment of the current invention relates to a spot-on composition comprising between about 0.25% and about 60% (w/w) pyrethroid, between about 0.01% and about 10% (w/w) of a combination of two or more macrocyclic lactones, between about 15% and about 45% (w/w) novaluron, and a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce the paresthesia caused by the pyrethroid to an animal. More specifically, this embodiment of present composition includes between about 10% and about 45% (w/w) pyrethroid, between about 0.05% and about 10% (w/w) of a combination of two or more macrocyclic lactones, between about 20% and about 40% (w/w) novaluron, and an effective amount of a paresthesia-reducing agent selected from the group consisting of purified diethylene glycol monoethyl ether, tocopherol succinate, tocopherol nicotinate, and combinations thereof. Most specifically, the spot-on composition of the present invention includes between about 15% and about 30% (w/w) pyrethroid, between about 0.05% and about 10% (w/w) of a combination of two or more macrocyclic lactones, between about 15% and about 35% (w/w) novaluron, and an effective amount of a paresthesia-reducing agent selected from the group consisting of purified diethylene glycol monoethyl ether, tocopherol succinate, tocopherol nicotinate, and combinations thereof. The macrocyclic lactones preferred in this embodiment are selected from the group consisting of ivermectin, selamectin, moxidectin, milbemycin, and combinations thereof.

A sixth embodiment of the current invention relates to a spot-on composition comprising between about 1% and about 20% (w/w) pyrethroid, between about 1% to about 20% (w/w) fipronil, between about 0.01% and about 10% (w/w) macrocyclic lactone, and a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce the paresthesia caused by the pyrethroid to an animal. More specifically, this embodiment of the spot-on composition includes between about 2% and about 10% (w/w) pyrethroid, between about 0.01% and about 0.2% (w/w) macrocyclic lactone, and an effective amount of a paresthesia-reducing agent selected from the group consisting of purified diethylene glycol monoethyl ether, tocopherol succinate, tocopherol nicotinate, and combinations thereof. Most specifically, the first embodiment of the present invention includes between about 4% and about 6% (w/w) pyrethroid, between about 0.05% and about 0.1% (w/w) macrocyclic lactone, and an effective amount of a paresthesia-reducing agent selected from the group consisting of purified diethylene glycol monoethyl ether, tocopherol succinate, tocopherol nicotinate, and combinations thereof. The macrocyclic lactone preferred in this embodiment is ivermectin.

A seventh embodiment of the current invention relates to a spot-on composition comprising between about 1% and about 20% (w/w) pyrethroid, between about 1% to about 20% (w/w) fipronil, between about 0.01% and about 10% (w/w) macrocyclic lactone, between about 1% and about 20% (w/w) insect growth regulator, and an effective amount of a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce the paresthesia caused by the pyrethroid to an animal. More specifically, this embodiment of present composition includes between about 2% and about 10% (w/w) pyrethroid, between about 0.01% and about 0.2% (w/w) macrocyclic lactone, between about 4% and about 15% (w/w) insect growth regulator, and an effective amount of a paresthesia-reducing agent selected from the group consisting of purified diethylene glycol monoethyl ether, tocopherol succinate, tocopherol nicotinate, and combinations thereof. Most specifically, the spot-on composition of the present invention includes between about 4% and about 6% (w/w) pyrethroid, between about 0.05% and about 0.1% (w/w) macrocyclic lactone, between about 7% and about 11% (w/w) insect growth regulator, and an effective amount of a paresthesia-reducing agent selected from the group consisting of purified diethylene glycol monoethyl ether, tocopherol succinate, tocopherol nicotinate, and combinations thereof. The macrocyclic lactone preferred in this embodiment is ivermectin.

An eighth embodiment of the current invention relates to a spot-on composition comprising between about 1% and about 20% (w/w) pyrethroid, between about 1% to about 20% (w/w) fipronil, between about 0.01% and about 10% (w/w) macrocyclic lactone, between about 1% and about 20% (w/w) insect growth regulator, and a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce the paresthesia caused by the pyrethroid to an animal. More specifically, this embodiment of present composition includes between about 2% and about 10% (w/w) pyrethroid, between about 1.5% and about 6% (w/w) macrocyclic lactone, between about 4% and about 15% (w/w) insect growth regulator, and an effective amount of a paresthesia-reducing agent selected from the group consisting of purified diethylene glycol monoethyl ether, tocopherol succinate, tocopherol nicotinate, and combinations thereof. Most specifically, the spot-on composition of the present invention includes between about 4% and about 6% (w/w) pyrethroid, between about 2% and about 4% (w/w) macrocyclic lactone, between about 7% and about 11% (w/w) insect growth regulator, and an effective amount of a paresthesia-reducing agent selected from the group consisting of purified diethylene glycol monoethyl ether, tocopherol succinate, tocopherol nicotinate, and combinations thereof. The macrocyclic lactone preferred in this embodiment is moxidectin.

A ninth embodiment of the current invention relates to a spot-on composition comprising between about 1% and about 20% (w/w) pyrethroid, between about 1% to about 20% (w/w) fipronil, between about 0.01% and about 10% (w/w) of a combination of two or more macrocyclic lactones, between about 1% and about 20% (w/w) insect growth regulator, and a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce the paresthesia caused by the pyrethroid to an animal. More specifically, this embodiment of present composition includes between about 2% and about 10% (w/w) pyrethroid, between about 0.05% and about 10% (w/w) of a combination of two or more macrocyclic lactones, between about 4% and about 15% (w/w) insect growth regulator, and an effective amount of a paresthesia-reducing agent selected from the group consisting of purified diethylene glycol monoethyl ether, tocopherol succinate, tocopherol nicotinate, and combinations thereof. Most specifically, the spot-on composition of the present invention includes between about 4% and about 6% (w/w) pyrethroid, between about 0.05% and about 10% (w/w) of a combination of two or more macrocyclic lactones, between about 7% and about 11% (w/w) insect growth regulator, and an effective amount of a paresthesia-reducing agent selected from the group consisting of purified diethylene glycol monoethyl ether, tocopherol succinate, tocopherol nicotinate, and combinations thereof. The macrocyclic lactones preferred in this embodiment are selected from the group consisting of ivermectin, selamectin, moxidectin, milbemycin, and combinations thereof.

A tenth embodiment of the current invention relates to a spot-on composition comprising between about 1% and about 20% (w/w) pyrethroid, between about 1% to about 20% (w/w) fipronil, between about 0.01% and about 10% (w/w) of a combination of two or more macrocyclic lactones, between about 15% and about 45% (w/w) insect growth regulator novaluron, and a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce the paresthesia caused by the pyrethroid to an animal. More specifically, this embodiment of present composition includes between about 2% and about 10% (w/w) pyrethroid, between about 0.05% and about 10% (w/w) of a combination of two or more macrocyclic lactones, between about 20% and about 40% (w/w) novaluron, and an effective amount of a paresthesia-reducing agent selected from the group consisting of purified diethylene glycol monoethyl ether, tocopherol succinate, tocopherol nicotinate, and combinations thereof. Most specifically, the spot-on composition of the present invention includes between about 4% and about 6% (w/w) pyrethroid, between about 0.05% and about 10% (w/w) of a combination of two or more macrocyclic lactones, between about 25% and about 35% (w/w) novaluron, and an effective amount of a paresthesia-reducing agent selected from the group consisting of purified diethylene glycol monoethyl ether, tocopherol succinate, tocopherol nicotinate, and combinations thereof. The macrocyclic lactones preferred in this embodiment are selected from the group consisting of ivermectin, selamectin, moxidectin, milbemycin, and combinations thereof.

In addition, the present invention further provides a method of eliminating and preventing heartworms, pest pupae and adults infestations on an animal, specifically a dog, the method comprising administering a localized cutaneous application of a spot-on composition of the present invention between the two shoulders of the animal in a volume sufficient to deliver a dose of the active components ranging from about 0.5 mg/kg to about 10 mg/kg of animal body weight.

Unless otherwise defined, all technical and scientific terms used herein have the same meaning as is commonly understood by one of skill in the art to which this invention belongs at the time of filing. If specifically defined, then the definition provided herein takes precedent over any dictionary or extrinsic definition. Further, unless otherwise required by context, singular terms shall include pluralities, and plural terms shall include the singular. Herein, the use of “or” means “and/or” unless stated otherwise. All patents and publications referred to herein are incorporated by reference.

### DETAILED DESCRIPTION OF THE INVENTION

The compositions provided herein are spot-on pesticide compositions that utilize combinations of certain active compounds to treat heartworm, insect, parasite, or tick and flea infestation of animals, specifically mammals (preferably dogs and cats), and also prevent future infestations by prolonged treatment efficacy that can last up to three months. As such, the compositions exterminate existing pests, and prevent those pests that survive from developing and reproducing. The compositions halt the growth cycle and prevent pests from laying additional eggs. The compositions of the current invention are useful in the treatment of many pests, especially heartworms, adult fleas and ticks, flea eggs and larvae, ear mites and sarcoptic mange found on domesticated animals. The compositions include low concentrations of a pyrethroid, a macrocyclic lactone, and may further comprise an additional pesticide (e.g., an insect growth-regulating compound, an N-arylpyrazole, or the like). In addition, the present invention is based in part on the finding that treatment of a host animal with compositions comprising a combination of a pyrethroid and a macrocyclic lactone results in dramatically higher kill rates within a shorter period of time of treatment than does treatment with an insect growth regulator, alone or combined, without the addition of a pyrethroid.

The present invention further comprises a novel paresthesia-reducing agent to reduce the paresthesia caused by the pyrethroid to an animal, wherein the paresthesia-reducing agent is selected from the group consisting of purified diethylene glycol monoethyl ether, tocopherol succinate, tocopherol nicotinate, and combinations thereof.

The spot-on compositions of the present invention comprise a pyrethroid compound. Generally, pyrethroids are a class of synthetic insecticides that are related to the naturally-occurring pyrethrins. Pyrethroids tend to be more effective than the natural pyrethrins, and less toxic to mammals. Pyrethroids are axonic poisons that work by keeping the sodium channels open in the neuronal membranes. The sodium channel consists of a membrane protein with a hydrophilic interior which permits sodium ions to enter and exit the membrane. When the sodium channels are kept open, the influx of sodium ions results in hyperexcitation and the pest becomes paralyzed. Suitable non-limiting examples of pyrethroids that may be used in the present invention include cyphenothrin, permethrin, cypermethrin, etofenprox, fenvalerate, cyfluthrin, and the like.

In one embodiment, the pyrethroid comprises between about 0.25% and about 60% (w/w) of the total weight of the spot-on composition. In some embodiments, the pyrethroid comprises about 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, or 0.25% (w/w), or any range thereof, of the spot-on composition. For example, the amount of a pyrethroid present in the spot-on composition may range from between about 10% to about 45% (w/w) of the total composition, and preferably ranges from about 15% to about 30% (w/w). In an exemplary embodiment, the amount of pyrethroid present in the spot-on composition effective for the treatment of pest infestations in mammals is about 20% (w/w) of the total composition.

The spot-on compositions of the present invention also comprise a macrocyclic lactone. Macrocyclic lactones are effective agents against both endoparasites and ectoparasites. Macrocyclic lactones are natural fermentation products of the fungus-like Streptomyces bacteria, including, but not limited to avermectin, abamectin, moxidectin, doramectin, ivermectin, and milbemycin, and the like, which share in common a large complex macrocyclic backbone.

In one embodiment the macrocyclic lactone is an avermectin compound. Avermectins are a group of chemically related potent anthelmintics and insecticides that are derivatives of a series 16-membered macrocyclic lactone. The naturally occurring avermectin compounds are produced by fermenting Streptomyces avermitilis, a soil actinomycete. The avermectin compounds are capable of blocking the transmittance of electrical activity in nerves and muscle cells by stimulating the release and binding of gamma-aminobutyric acid (GABA) at nerve endings. Different avermectins have a major (B₁ₐ-component) and minor (B_{1b}-component) component usually in ratios of 80:20 to 90:10. Anthelmintics derived from the avermectins include ivermectin, selamectin, doramectin, and abamectin.

The avermectin compound typically comprises between about 0.01% and about 10% (w/w) of the spot-on composition. In some embodiments, the avermectin comprises about 10%, 9%, 8%, 7%, 6%, 5%, 4%, 3.5%, 3%, 2.5%, 2%, 1.5%, 1%, 0.5%, 0.25%, 0.1%, 0.09%, 0.08%, 0.07%, 0.06%, 0.05%, 0.04%, 0.03%, 0.02%, or 0.01% (w/w) of the spot-on composition. For example, the amount of avermectin compound present in the spot-on composition may range from between about 0.01% to about 8% (w/w) of the total composition, and preferably ranges from about 0.05% to about 5% (w/w). In another embodiment, the amount of avermectin compound present in the spot-on composition may range from about 0.1% to about 5% (w/w) of the total composition. In a further embodiment, the amount of avermectin compound present in the spot-on composition may range from about 0.2% to about 1.5% (w/w) of the total composition. In still another embodiment, the amount of avermectin compound present in the spot-on composition may range from between about 1.5% to about 3% (w/w) of the total composition. In a further embodiment, the amount of avermectin compound present in the spot-on composition may range from between about 0.06% to about 0.09% (w/w) of the total composition.

In one embodiment, the avermectin in the composition is ivermectin. Ivermectin is the combination of two dehydrogenated avermectins (22,23-dihydroavermectin B1a+22,23-dihydroavermectin B1b), and is a broad-spectrum antiparasitic avermectin medicine.

In another embodiment, the avermectin compound present in the composition is selamectin (C43H63NO11). Selamectin disables parasites by replacing glutamate in their muscle synapses. Selamectin interferes the interaction between glutamate and its receptors that open chloride channels into the muscle, and activates the chloride current without desensitization, allowing chloride ions to enter the nerve cells and causing neuromuscular paralysis, impaired muscular contraction, and eventual death of parasites.

Alternatively, the spot-on pesticide composition of the current invention may comprise a macrocyclic lactone that is a moxidectin or a milbemycin compound. Moxidectin is a semisynthetic derivative of nemadectin which is produced by fermentation by Streptomyces cyano-griseus. Both moxidectin and milbemycin treat and control some of the most common internal and external parasites by selectively binding to parasites' glutamate-gated chloride ion channels. These channels are vital to the function of invertebrate nerve and muscle cells. When moxidectin or milbemycin binds to the channels, it disrupts neurotransmission, resulting in paralysis and death of the parasite.

The moxidectin compound typically comprises between about 1% and about 5% (w/w) of the spot-on composition. In some embodiments, the moxidectin comprises about 5%, 4.5%, 4%, 3.5%, 3%, 2.5%, 2%, 1.5%, or 1% (w/w) of the spot-on composition. For example, the amount of a moxidectin present in the spot-on composition may range from between about 1% to about 4.5% (w/w) of the total composition, and preferably ranges from about 1.5% to about 4% (w/w). In another embodiment, the amount of a moxidectin present in the spot-on composition may range from about 2% to about 5% (w/w) of the total composition. In a further embodiment, the amount of a moxidectin present in the spot-on composition may range from about 2% to about 4% (w/w) of the total composition.

As an alternative to moxidectin, milbemycin may be used and typically comprises between about 1% and about 5% (w/w) of the spot-on composition. In some embodiments, the milbemycin comprises about 5%, 4.5%, 4%, 3.5%, 3%, 2.5%, 2%, 1.5%, or 1% (w/w) of the spot-on composition. For example, the amount of a milbemycin present in the spot-on composition may range from between about 1% to about 4.5% (w/w) of the total composition, and preferably ranges from about 1.5% to about 4% (w/w). In another embodiment, the amount of a milbemycin present in the spot-on composition may range from about 2% to about 5% (w/w) of the total composition. In a further embodiment, the amount of a milbemycin present in the spot-on composition may range from about 2% to about 4% (w/w) of the total composition.

The compositions of the present invention may include at least one macrocyclic lactone, or alternatively, may comprise a combination of two or more macrocyclic lactones.

The spot-on pesticide compositions of the current invention may additionally include an insect growth regulator (IGR). IGRs are not effective in killing pre-existing pests; they prevent reproduction and further infestation. An IGR is generally a compound that is capable of disrupting the growth and development of pest species, so that the pest cannot mature and reproduce. The IGR typically comprises less than about 20% (w/w) of the total weight of the spot-on composition. In some embodiments, the IGR comprises about 19%, 18%, 17%, 16%, 15%, 14%, 13%, 12%, 11%, 10%, 9%, 8%, 7%, 6%, 5%, 4%, 3%, 2%, 1%, 0.1%, or 0% (w/w) of the spot-on composition. For example, the amount of IGR present in the spot-on composition may range from between 1% to about 20% (w/w) of the total composition weight, and preferably the IGR ranges from between about 2% to about 15% (w/w) of the total composition. In another embodiment, the amount of IGR present in the spot-on composition may range from about 4% to about 12% (w/w) of the total composition. In a further embodiment, the amount of IGR present in the spot-on composition may range from about 7% to about 14% (w/w) of the total composition. In still another embodiment, the amount of IGR may range from between about 8% to about 12% (w/w) of the total composition. In an additional embodiment, the amount of IGR may range from between about 2% to about 9% (w/w) of the total composition. In a further embodiment, the amount of IGR may range from between about 3% to about 5% (w/w) of the total composition. IGRs may include, but are not limited to juvenile hormone mimics, chitin synthesis inhibitors, and the like.

Suitable non-limiting examples of insect growth regulators include bistrifluron, buprofezin, chlorfluazuron, cyromazine, diflubenzuron, flucycloxuron, flufenoxuron, hexaflumuron, lufenuron, novaluron, noviflumuron, penfluron, teflubenzuron, triflumuron, epofenonane, fenoxycarb, hydroprene, kinoprene, methoprene, pyriproxyfen, triprene, and combinations thereof. In a preferred embodiment, the insect growth regulator is S-methoprene. Generally, methoprene is a racemic mixture of the R- and S-enantiomers of the compound, however, only the S-enantiomer is active as a juvenile hormone analog. A juvenile hormone analog exerts a therapeutic effect by mimicking the natural juvenile hormones found within pests. Juvenile hormone must be absent for a pupa to molt to an adult, so methoprene treated larvae are unable to successfully develop from pupa to an adult pest. This action breaks the natural life cycle of the pest, preventing it from maturing and reproducing. S-methoprene is also known as isopropyl (2E, 4E, 7S)-11-methoxy-3,7,11-trimethyl-2,4-dodecadienoate. S-methoprene is available in a variety of commercial products and is useful in controlling long-term pest infestation, while other active components are primarily effective in the immediate, short-term elimination of pests. The pest kill time for treatment with S-methoprene will vary depending on the typical duration of life for the species being treated. Unlike some other compounds, S-methoprene is generally considered non-toxic to humans, which has led to its use in the treatment of well cisterns and a number of food items, including meat, milk, mushrooms, peanuts, rice, and cereals. In an exemplary embodiment, the concentration of S-methoprene present in the spot-on composition effective for the treatment of pest infestations in animals is about 8.8% (w/w) of the total composition.

In an alternative embodiment (i.e. those embodiments of the present invention that do not contain S-methoprene), the insect growth regulator may be the juvenile hormone analog pyriproxyfen, also known as 4-phenoxyphenyl 2-(2-pyridyloxy)propyl ether and NylarTM. In an exemplary embodiment, the amount of pyriproxyfen present in the spot-on composition effective for the treatment of pest infestations in animals is about 2% (w/w) of the total composition.

In an alternative embodiment (i.e. those embodiments of the present invention that do not contain S-methoprene or pyriproxyfen), the insect growth regulator may be the novaluron (C₁₇H₉ClF₈N₂O₄), also known as N-[[[3-chloro-4-[1,1,2-trifluoro-2-(trifluoromethoxy)ethoxy]phenyl]amino]carbonyl]-2,6-difluorobenzamide. In an exemplary embodiment, the concentration of novaluron present in the spot-on composition effective for the treatment of pest infestations in animals may be as high as between about 15% and 45% (w/w).

The spot-on compositions of the present invention may optionally include the N-arylpyrazole compound known as fipronil. Fipronil is a phenylpyrazole acaricide with efficacy against a broad spectrum of tick species and was first disclosed in U.S. Pat. No. 5,232,940. Fipronil achieves its efficacy by disrupting the central nervous system by blocking the passage of chloride ions through the GABA receptor and glutamate-gated chloride channels (GluCl), components of the central nervous system. This disruption causes hyperexcitation of contaminated nerves and muscles, which results in eventual death. The compound is a slow-acting acaricide, and as such, can be used to target not only the host, but also other ticks in which the host comes in contact. Fipronil is also known as 5-amino-1-[2,6-dichloro-4-(trifluoromethyl)phenyl]-4-[(1-R,S)(trifluoromethyl)sulfinyl]-1H-pyrazole-3-carbonitrile, 5-amino-1-(2,6-dichloro-α,α,α-trifluoro-p-tolyl)-4- [(trifluoromethyl) sulfinyl] pyrazole-3-carbonitrile, and fluocyanobenpyrazole [CAS No. 120068-37-3]. Fipronil is generally available as either a liquid or solid crystalline substance or powder. Fipronil typically comprises between about 1% and about 20% (w/w) of the total weight of the spot-on composition. In some embodiments, fipronil comprises about 20%, 19%, 18%, 17%, 16%, 15%, 14%, 13%, 12%, 11%, 10%, 9%, 8%, 7%, 6%, 5%, 4%, 3%, 2%, or 1% (w/w) of the spot-on composition. For example, the amount of fipronil present in the spot-on composition may range from between about 5% to about 15% (w/w) of the total composition, and preferably ranges from between about 7% and about 12% (w/w). Most preferably, the amount of fipronil present in the spot-on composition may range from between about 8% and about 11% (w/w) of the total composition. In an exemplary embodiment, the amount of fipronil present in the composition is 9.8% (w/w) of the total composition. The spot-on compositions of the present invention do not additionally include crystallization inhibitors.

The spot-on compositions of the present invention also comprise a paresthesia-reducing agent selected from the group consisting of purified diethylene glycol monoethyl ether, tocopherol succinate, tocopherol nicotinate, and combinations thereof. Many of the commercially-available compositions that incorporate a pyrethroid, including cyphenothrin, have reported that the animals suffer from adverse effects including paraesthesia (a skin sensation that generally comprises feelings of prickling, itching, and tingling). However, it has been found that inclusion of a paresthesia-reducing agent into the spot-on composition reduces or eliminates the undesirable adverse effects associated with treatment regimens that include pyrethroids. The paresthesia-reducing agent generally comprises between about 40% to about 90% (w/w) of the spot-on composition. In some embodiments, the paresthesia-reducing agent comprises 90%, 85%, 80%, 75%, 70%, 65%, 60%, 55%, 50%, 45%, or 40% (w/w) of the total composition. In an embodiment, the amount of paresthesia-reducing agent present in the spot-on composition preferably ranges from between about 60% to about 80% (w/w) of the composition. In another embodiment, the amount of paresthesia-reducing agent present in the spot-on composition ranges from between about 60% to about 75% (w/w) of the total composition. In an additional embodiment, the amount of paresthesia-reducing agent present in the spot-on composition ranges from between about 70% to about 85% (w/w) of the total composition. In still another embodiment, the amount of organic solvent in the spot-on composition ranges from between about 60% to about 70% (w/w).

In an alternative embodiment, the spot-on compositions of the present invention may comprise an organic solvent rather than a paresthesia-reducing agent. Generally, the organic solvent is defined as a carbon-containing chemical that is capable of dissolving a solid, liquid, or a gas. Although one skilled in the art will appreciate that a wide variety of solvents may be incorporated into the current invention, the solvents should generally have a dielectric constant ranging from about 1 to 40, a low boiling point (less than 100° C.), have a density less than the density of water (less than 1.0 at 20° C.), and generally be soluble with water. Suitable examples of organic solvents that may be used in the present invention include, but are not limited to, acetyltributyl citrate, fatty acid esters such as dimethyl ester, diisobutyl adipate, acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, diethylene glycol monoethyl ether, monomethylacetamide, dipropylene glycol monomethyl ether, liquid polyoxyethylene glycols, propylene glycol, 2-pyrrolidones such as N-methylpyrrolidone, diethylene glycol monoethyl ether, ethylene glycol, diethyl phthalate, ethoxydiglycol, or combinations thereof. In a preferred embodiment, the organic solvent comprises diethylene glycol monoethyl ether.

An organic solvent may comprise between about 55% to about 85% (w/w) of the spot-on composition. In some embodiments, the organic solvent comprises 85%, 80%, 75%, 70%, 65%, 60%, or 55% (w/w) of the total composition. For example, the amount of organic solvent present in the spot-on composition may range from between about 60% to about 80% (w/w) of the composition. In another embodiment, the amount of organic solvent in the spot-on composition may range from between about 60% to about 75% (w/w) of the total composition. In an additional embodiment, the amount of organic solvent in the spot-on composition may range from between about 70% to about 80% (w/w) of the total composition. In still another embodiment, the amount of organic solvent in the spot-on composition may range from between about 60% to about 70% (w/w).

In addition to an organic solvent, the spot-on composition may further include an antioxidant. An antioxidant can generally be defined as a compound capable of slowing or preventing the oxidation of other molecules. Oxidation is a chemical reaction that transfers electrons from the original substance to an oxidizing agent. Oxidation reactions can produce free radicals, which start chain reactions that damage cells. Antioxidants terminate these chain reactions by removing free radical intermediates, and inhibit other oxidation reactions by being oxidized themselves. Within the spot-on composition, the antioxidant acts as a stabilizer, preventing the various components from degrading by oxidation processes.

If an antioxidant is incorporated into the current invention it should generally be miscible with the organic solvents described herein. It is preferred that the antioxidant does not cause irritation to the skin of an animal, specifically a dog or cat, when applied to the animal's skin. In addition, the antioxidant may be natural or synthetic. Suitable antioxidants include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-carotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., Ionox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivatives, vitamin Q10, wheat germ oil, zeaxanthin, or combinations thereof. One skilled in the art will appreciate that the antioxidants incorporated into the composition (including those listed herein) encompass all potential salt and ester forms of the antioxidants in addition to the pure forms of the compound. Preferably, the antioxidant comprises a vitamin E compound such as tocopherol acetate, tocopherol linoleate, tocopherol nicotinate, tocopherol succinate, ascorbyl tocopherol phosphate, dioleyl tocopherol methylsilanol, tocophersolan, and tocopherol linoleate/oleate. In an exemplary embodiment, the antioxidant comprises tocopherol nicotinate.

The antioxidant may comprise less than about 10% (w/w) of the total spot-on composition. In some embodiments, the antioxidant comprises about 9%, 8%, 7%, 6%, 5%, 4%, 3%, 2%, 1%, 0.1%, or 0% (w/w) of the total composition. For example, the amount of antioxidant present in the spot-on composition may range from between 2% to about 10% (w/w) of the total composition, and preferably the antioxidant ranges from between about 3% to about 6% (w/w) of the total composition. In a further embodiment, the amount of antioxidant present in the spot-on composition may range from between about 4% to about 6% (w/w) of the total composition. In an exemplary embodiment, the amount of antioxidant present in the composition is about 5.3% (w/w).

The spot-on composition may further include inactive excipients that are added to the composition as a result of their incorporation into the individual active components. For instance, the cyphenothrin and ivermectin components of the composition may be provided in a 95% solution, meaning that 95% of the composition volume is active compound including cyphenothrin and ivermectin, and the remaining 5% constitutes inactive excipients that are consequently introduced into the composition, as such the pesticide may not be 100% pure concentrate and may be purchased with other constituents. One skilled in the art will recognize that the inactive excipients include, but are not limited to binders, fillers, non-effervescent disintegrants, effervescent disintegrants, preservatives, diluents, lubricants, pH modifiers, stabilizers, and the like. It should, however, be understood that the inactive excipients are typically incorporated as a portion of the active ingredient components and comprise a small percentage (generally less than 1%) of the total spot-on composition volume, generally not affecting the physical characteristics of the spot-on composition.

It should be understood that the active components of the spot-on composition may be provided in the form of pure concentrate (100% concentration) or a diluted composition with additional excipients in the dosage form (i.e. the amount of active ingredient in the composition is less than or equal to 99.99%, and the remainder consists of inactive excipients). One of skill in the art will appreciate that the volume of active component added to the spot-on composition will need to be adjusted to account for the dilution and to ensure the end spot-on composition comprises the appropriate final concentration of each of the active components. One of skill in the art will also appreciate that the various components of the spot-on composition may be provided in a variety of dosage forms including, but not limited to powder, briquettes, liquid solution or suspension, pellets, emulsion, aerosol, cream, gel, ointment, and the like.

Additionally, the spot-on pesticide composition of the current invention can be produced by contacting the various active components of the spot-on composition with one another to produce a spot-on formulation suitable for application to an animal's skin. It should be understood that the current invention encompasses a variety of physical formulations; however, the spot-on compositions of the current invention are generally directed to liquid solutions and suspensions. The formulations of the present invention may be prepared by standard techniques known in the art. For instance, in one embodiment where the desired spot-on formulation is a liquid solution, the composition is produced by bringing the pyrethroid and avermectin components into contact with a paresthesia-reducing agent and then gently heating and stirring the components until dissolved. A person having ordinary skill in the art will appreciate that the various components of the spot-on composition may be contacted and mixed with one another in any order desired, so long as solution is adequately stirred and mixed.

The physical characteristics of the spot-on composition may vary depending upon the physical characteristics desired. However, the spot-on composition should be capable of application to the skin of an animal and provide adequate stasis to allow the active components of the spot-on composition to be absorbed by the host animal. Preferably, the spot-on compositions of the present invention have low viscosity. Viscosity is the measurement of flow resistance due to internal friction within the fluid, and is measured in centistokes (cSt). A lower cSt measurement means the fluid will flow with less resistance, because of minimal molecular friction within the fluid. The lower the viscosity the faster the fluid will flow. High viscosity substances are liquids that are thick and gelatinous in nature with slow flow. Low viscosity substances exhibit a fast flow with an example being water at room temperature (water at 20° C. has a viscosity of about 1 cSt; 1cSt=1 mm²/second). The spot-on compositions of the present invention typically have a viscosity ranging from about 0.01 mm²/second to about 100 mm²/second. In a more preferred embodiment, the spot-on composition has a viscosity ranging from about 1 mm²/second to about 30 mm²/second. In a further preferred embodiment, the spot-on composition has a viscosity ranging from about 4 mm²/second to about 20 mm²/second.

A basic spot-on composition of the present invention includes a pyrethroid at a concentration ranging between about 0.25% and about 60% (w/w), a macrocyclic lactone at a concentration ranging between about 0.01% and about 5% (w/w), and an effective amount of a paresthesia-reducing agent selected from the group consisting of purified diethylene glycol monoethyl ether, tocopherol succinate, tocopherol nicotinate, and combinations thereof. For the basic spot-on composition, it is preferable to use between about 15% to about 30% (w/w) pyrethroid, about 1% to about 5% (w/w) macrocyclic lactone, and about 50% to about 80% (w/w) paresthesia-reducing agent, depending on the type of animal to be treated. The basic spot-on composition may also additionally include an IGR at a concentration ranging between 1% and about 20% (w/w) of the composition, preferably at a concentration ranging between about 2% to about 12% (w/w) of the total composition. More preferably, the IGR is present in the composition at a concentration ranging between 4% and 12% (w/w) of the total composition. In addition, the basic spot-on composition may further include an antioxidant at a concentration ranging between 1% and about 10% (w/w) of the total composition, preferably at a concentration ranging between about 4% and about 6% (w/w) of the total composition.

The present invention further embodies a method of killing heartworm, pest pupae and adults on an animal comprising administering a localized cutaneous application between the shoulders of the animal, a spot-on composition comprising between about 0.25% to about 60% (w/w) pyrethroid and between about 0.01% to about 5% (w/w) macrocyclic lactone. The method of the using the composition of the present invention preferably involves the localized administration of the basic spot-on composition, which composition preferably comprises between about 15% to about 30% (w/w) pyrethroid, about 0.05% to about 5% (w/w) macrocyclic lactone, and may additionally include between about 4% to about 12% (w/w) of an IGR.

The compositions and method according to this invention are intended for application to animals, in particular dogs and cats, and are generally applied by deposition onto the skin (“spot-on” or “pour-on” application). Treatment typically comprises a localized application over a surface area of less than 10 cm², especially of between 5 and 10 cm². Generally, the spot-on composition should be applied to an area where the animal cannot lick the application area, as licking of the application area may lead to transient adverse effects, such as excessive salivation. In particular, application is preferred at two points and preferably localized between the animal's shoulders. After the spot-on composition has been applied, the composition diffuses, in particular over the animal's entire body, and then dries without crystallizing or modifying the appearance (in particular absence of any whitish deposit or dusty appearance) or the feel of the animal's fur. Further, the method of the current invention is directed to application of the spot-on composition to the skin of the animal every four weeks to ensure continuous treatment and prevention of pest infestation. Typically, the active constituents are applied to the host animal together in a single formulation.

In another embodiment, the method of killing insects is carried out such that the spot-on composition is applied in a volume sufficient to deliver a dosage of the active pyrethroid component ranging from about 0.1 mg/kg to about 40 mg/kg of host animal body weight. In a preferred embodiment, the dose of pyrethroid ranges from about 0.5 mg/kg to about 20 mg/kg of host animal body weight. In a more preferred embodiment, the spot-on composition application comprises a volume sufficient to deliver a pyrethroid dose ranging from about 0.5 mg/kg to about 10 mg/kg of host animal body weight.

In a further embodiment, the method of killing insects is carried out such that the spot-on composition further comprises an IGR, and is applied in a volume sufficient to deliver a dosage of the insect growth regulating active component ranging from about 0.1 mg/kg to about 40 mg/kg of host animal body weight. In a preferred embodiment, the dose of insect growth regulator ranges from about 0.2 mg/kg to about 20 mg/kg of host animal body weight. In a more preferred embodiment, the spot-on composition application comprises a volume sufficient to deliver an insect growth regulator dose ranging from about 0.5 mg/kg to about 10 mg/kg of host animal body weight. The composition provided herein is safe for use in puppies as young as 6 weeks old, breeding and nursing animals, and avermectin-sensitive collies.

One of skill in the art will understand that the dosage ranges provided above are approximate values that may vary within a broad range. The variance in dose is due to the fact that, in practice, the spot-on composition will be administered in defined doses and volumes to animals within a certain range of weights. As a result, the dosage actually applied to the animal may vary by a factor ranging from 0.1 to 10 relative to the preferred dose, without imparting any additional risks pertaining to toxicity or decreased efficacy.

Although the components of the composition are effective against a wide variety of pests and parasites, the composition is especially developed for the treatment of fleas (including the Ctenocephalides species), ticks (the Rhipecephalus, Ixodes, and Trichodectes species), heartworms (including the Dirofilaria immitis species), ear mites (including the Otodectes cynotis species), and Sarcoptes spp. that causes mange in animal. Other parasites that can be treated by the composition include: hookworms (including Ancylostoma species and Uncinaria species), roundworms (including Toxascaris lenoina, Toxocara canis, and Toxocara cati species), whipworms (including Trichuris vulpis and Trichuris campanula species), and tapeworms (including Dipylidium caninum, Taenia species, Echinococcus granulosus and Echinococcus multiocularis, Diphyllobothrium latum and Spirometra mansonoides species).

Furthermore, the frequency of application may be varied according to the needs of the individual animal, as well as the severity of infestation. The treatment of parasites may be repeated as often as once weekly, or may be reserved for one-time acute treatments of pest infestation or flare-ups. In one embodiment of the current invention, the treatment of parasites may be repeated about every four weeks, five weeks, or six weeks. In another embodiment, the spot-on composition is applied to the host animal for a one-time treatment of the pest infestation. In an embodiment of the current invention, parasites are treated at a frequency ranging from one to four weeks, with treatment every two weeks being preferred. In another embodiment, the spot-on composition is applied on a one-time basis for the treatment of parasites infestation.

Although the invention described herein is susceptible to various modifications and alternative iterations, specific embodiments thereof have been described in greater detail above. It should be understood, however, that the detailed description of the spot-on composition is not intended to limit the invention to the specific embodiments disclosed. Rather, it should be understood that the invention is intended to cover all modifications, equivalents, and alternatives falling within the spirit and scope of the invention as defined by the claim language.

### DEFINITIONS

As used herein, the terms “about” and “approximately” designate that a value is within a statistically meaningful range. Such a range can be typically within 20%, more typically still within 10%, and even more typically within 5% of a given value or range. The allowable variation encompassed by the terms “about” and “approximately” depends on the particular system under study and can be readily appreciated by one of ordinary skill in the art.

As used herein, the term “w/w” designates the phrase “by weight” and is used to describe the concentration of a particular substance in a mixture or solution.

As used herein, the term “mL/kg” designates milliliters of composition per kilogram of body weight.

As used herein, the term “treatment” or “treating” of a condition, such as pest infestation, includes inhibiting an existing condition or arresting its development; or ameliorating or causing regression of the condition. The term “preventing” or “prevention” of a condition, such as insect or pest infestation, includes substantially blocking or inhibiting the development or growth of a condition before it starts. Compositions that treat or prevent infestations herein will preferably exhibit at least 90% efficacy.

As used herein, the term “pesticide” or “pesticidal” refers to an agent or a composition comprising an agent that is capable of preventing, reducing or eliminating pest infestations. Preferred pesticides of the present invention include cyphenothrin and ivermectin.

As used herein, the term “insect growth regulator” or “IGR” refers to an agent that is capable of interrupting or inhibiting the life cycle of a pest such that the pest never matures into an adult and becomes incapable of reproducing. Preferred IGRs of the present invention include S-methoprene and pyriproxyfen.

As used herein, the term “animal” refers to a mammal, specifically a companion animal, including but not limited to dogs, cats, rabbits, ferrets, horses, and hamsters.

As used herein, the term “pest” and “insect” refers to any ectoparasite, including but not limited to fleas, ticks, flies, keds, mosquitoes, and mites.

“Paresthesia” as used herein and in the appended claims is defined as primarily a condition that results in a feeling (burning, tingling, and/or pricking sensation) of the skin.

As used herein, the term “paresthesia-reducing agent” refers to an agent or combination of agents that reduces or eliminates paresthesia and is selected from the group consisting of purified diethylene glycol monoethyl ether, tocopherol nicotinate and tocopherol succinate, and combinations thereof.

### EXAMPLES

### Example 1

### Spot-On Formulation

A spot-on formulation was prepared in accordance with Table 1.

TABLE 1
Formulation

| | Concentration |
|---|---|
| Ingredients | (%) |
| Cyphenothrin (94%) | 20.000 |
| Moxidectin | 2.500 |
| Methoprene (95.5%) | 9.210 |
| Purified diethylene glycol monoethyl ether | 62.990 |
| (Transcutol CG) | |
| Vitamin E Nicotinate (tocopherol) | 5.300 |
| TOTAL: | 100.000 |

The purified diethylene glycol monoethyl ether and the tocopherol nicotinate were charged to a vessel and heated to a temperature of 50° C. (about 1 hour). Once heated, the cyphenothrin, the moxidectin, and the methoprene were charged to the vessel and all components were mixed until a homogenous solution was formed (about 1 hour).

The composition prepared exhibited at least 90% efficacy against pests (including ticks) for an extended period of time.

What is claimed is:

1. A spot-on pesticidal composition comprising between about 0.25% to about 60% (w/w) pyrethroid and about 0.01% to about 10% (w/w) macrocyclic lactone.

2. The spot-on pesticidal composition of claim 1, wherein the macrocyclic lactone is selected from the group consisting of ivermectin, selamectin, moxidectin, milbemycin, and combinations thereof.

3. The spot-on pesticidal composition of claim 1, comprising about 10% to about 45% (w/w) pyrethroid.

4. The spot-on pesticidal composition of claim 1, comprising about 15% to about 30% (w/w) pyrethroid and about 0.05% to about 5% (w/w) macrocyclic lactone.

5. The spot-on pesticidal composition of claim 1, wherein the pyrethroid is cyphenothrin.

6. The spot-on pesticidal composition of claim 1, further comprising about 1% to about 20% (w/w) of an insect growth regulator.

7. The spot-on composition of claim 6, comprising about 2% to about 15% (w/w) of an insect growth regulator.

8. The spot-on composition of claim 1, wherein the insect growth regulator comprises pyriproxyfen, S-methoprene, or novaluron.

9. The spot-on composition of claim 1, further comprising an effective amount of a paresthesia-reducing agent selected from the group consisting of purified diethylene glycol monoethyl ether, tocopherol succinate, tocopherol nicotinate, and combinations thereof.

10. A spot-on pesticidal composition comprising between about 0.25% to about 60% (w/w) pyrethroid, about 0.01% to about 10% (w/w) macrocyclic lactone, and about 1% to about 20% (w/w) of an insect growth regulator.

11. The spot-on composition of claim 10, wherein the composition comprises about 2% to about 15% (w/w) of an insect growth regulator.

12. The spot-on composition of claim 10, wherein the insect growth regulator comprises pyriproxyfen, S-methoprene, or novaluron.

13. A spot-on pesticidal composition comprising between about 0.25% to about 60% (w/w) pyrethroid, about 0.01% to about 10% (w/w) macrocyclic lactone, and an effective amount of a paresthesia-reducing agent selected from the group consisting of purified diethylene glycol monoethyl ether, tocopherol succinate, tocopherol nicotinate, and combinations thereof.

14. The spot-on composition of claim 13, wherein the paresthesia-reducing agent is present in an amount from between about 40% and about 90% (w/w) of the composition.

15. A spot-on pesticidal composition comprising about 1% to about 20% (w/w) pyrethroid, 1% to about 20% (w/w) fipronil, and about 0.01% to about 10% (w/w) macrocyclic lactone.

16. The spot-on pesticidal composition of claim 15, wherein the macrocyclic lactone is selected from the group consisting of ivermectin, selamectin, moxidectin, milbemycin, and combinations thereof.

17. The spot-on pesticidal composition of claim 15, wherein the pyrethroid is cyphenothrin.

18. The spot-on composition of claim 15, further comprising about 1% to about 20% (w/w) of an insect growth regulator.

19. A spot-on pesticidal composition comprising about 1% to about 20% (w/w) pyrethroid, 1% to about 20% (w/w) fipronil, about 0.01% to about 10% (w/w) macrocyclic lactone, and about 1% to about 20% (w/w) of an insect growth regulator.

20. A method of killing heartworm, insect and pest pupae and adults on an animal, which method comprises administering a localized cutaneous application between the shoulders of the animal, a spot-on composition comprising about 0.25% to about 60% (w/w) pyrethroid and about 0.01% to about 10% (w/w) macrocyclic lactone.

21. The method of claim 20, wherein the macrocyclic lactone is selected from the group consisting of ivermectin, selamectin, moxidectin, milbenycin and combinations thereof.

22. The method of claim 20, wherein the spot-on composition comprises about 10% to about 45% (w/w) of a pyrethroid.

23. The method of claim 20, wherein the spot-on composition comprises about 15% to about 30% (w/w) of a pyrethroid.

24. The method of claim 20, wherein the animal is a mammal.

25. The method of claim 24, wherein the mammal comprises a dog or a cat.

26. The method of claim 20, wherein the spot-on composition further comprises an insect growth regulator.

27. The method of claim 26, wherein the insect growth regulator is pyriproxyfen or S-methoprene.

28. The method of claim 26, wherein the insect growth regulator is present in the composition at a concentration of between about 2% to about 15% (w/w).

29. The method of claim 26, wherein the insect growth regulator is present in the composition at a concentration of between about 8% to about 12% (w/w).

30. The method of claim 25, wherein the insect growth regulator is present in the composition at a concentration of between about 3% to about 5% (w/w).

31. The method of claim 20, wherein the spot-on composition additionally comprises a paresthesia-reducing agent selected from the group consisting of purified diethylene glycol monoethyl ether, tocopherol succinate, tocopherol nicotinate, and combinations thereof, wherein the paresthesia-reducing agent is present in an amount effective to reduce or eliminate the paresthesia caused by the synthetic pyrethroid to mammals.

32. The method of claim 20, wherein the composition is administered for treating and preventing heartworm, insect and pest pupae and adults, ear mites, and Sarcoptes spp. that causes mange, hookworms, roundworms, whipworms, and tapeworms.

33. A method of killing heartworm, insect and pest pupae and adults on an animal, which method comprises administering a localized cutaneous application between the shoulders of the animal, a spot-on composition comprising about 1% to about 20% (w/w) pyrethroid, aboutl% to about 20% (w/w) fipronil, and about 0.01% to about 10% (w/w) macrocyclic lactone.

34. The method of claim 33, wherein the macrocyclic lactone is selected from the group consisting of ivermectin, selamectin, moxidectin, milbenycin and combinations thereof.

35. The method of claim 33, wherein the spot-on composition comprises about 4% to about 6% (w/w) of a pyrethroid.

36. The method of claim 33, wherein the spot-on composition further comprising about 1% to about 20% (w/w) of an insect growth regulator.
